(19) United States
(12) Patent Application Publication
(10) Pub. No.: US 2024/0238179 A1
(43) Pub. Date: Jul. 18, 2024

(54) SOLID ANHYDROUS COMPOSITION FOR CARING FOR AND/OR MAKING UP KERATIN MATERIALS

(71) Applicant: L'OREAL, Paris (FR)

(72) Inventors: Zheng GONG, Shanghai (CN); Tu LUAN, Shanghai (CN)

(73) Assignee: L'OREAL, Paris (FR)

(21) Appl. No.: 18/554,855

(22) PCT Filed: Apr. 30, 2021
(86) PCT No.: PCT/CN2021/091514

§ 371 Date: Oct. 11, 2023

(51) Int. Cl. A61K 8/37; A61K 8/02; A61K 8/25; A61K 8/31; A61K 8/34; A61K 8/92; A61Q 1/06

(52) U.S. Cl. CPC A61K 8/375; A61K 8/0229; A61K 8/25; A61K 8/31; A61K 8/345; A61K 8/922; A61Q 1/06; A61K 2800/31; A61K 2800/43; A61K 2800/48

(57) ABSTRACT

A solid anhydrous composition for caring for and/or making up keratin materials comprises a) 2 wt. % or more of at least one hydrophilic moisturizer, relative to the total weight of the composition; b) at least one non-ionic surfactant selected from glyceryl fatty acid monoesters and polyglyceryl fatty acid esters; c) polyethylene wax; and e) at least one oil. A cosmetic process for caring for and/or making up keratin materials comprises applying the solid anhydrous composition according to the present invention to the keratin materials.

### TECHNICAL FIELD

The present invention relates to a composition for caring for and/or making up keratin materials. More particularly, the present invention relates to a solid anhydrous composition for caring for and/or making up keratin materials. The present invention also relates to a cosmetic process for caring for and/or making up keratin materials.

### BACKGROUND ART

Lipsticks have been used for many years to accentuate the positive aspects of the wearer's lips. Lipsticks are capable of altering the apparent facial characteristics of the wearer. Besides altering the shape of the lips, lipsticks can be made in a great number of colors and shades to promote a desired effect or express the mood of the wearer.

To date, some prior art documents relating to cosmetic solid compositions for making up and/or caring for the skin and/or the lips have been published.

For example, WO 2013/191300 discloses a solid cosmetic composition for making up and/or caring for the skin and/or the lips, comprising, in a physiologically acceptable medium, at least one fatty phase comprising:
from 5 to 30% by weight of a non-volatile apolar oil, or a mixture thereof, relative to the total weight of the composition,
from 43 to 90% by weight of the total content of a nonvolatile silicone oil relative to the total weight of the composition, wherein at least one of said non-volatile silicone oil(s) is a non-volatile phenylated silicon oil, and
from 3 to 30% by weight of a wax, or a mixture thereof, relative to the total weight of the composition.

Lipsticks with high amount of pigments usually show the disadvantage of not hydrating or moisturizing enough. In order to improve this disadvantage, many efforts have been made.

One of the good solutions is to incorporate a high amount of polyols such as glycerin as efficient humectants in the anhydrous solid product. However, due to the hygroscopicity of glycerin, such stick compositions are prone to having a sweating problem (i.e., a number of droplets appear on the lipstick surface) after a temperature and/or humidity fluctuation, which is regarded as a quality issue by consumers.

Therefore, there is a need to develop a moisturizing lipstick, which shows good stability (i.e. anti-sweating performance).

### SUMMARY OF THE INVENTION

An object of the present invention is thus to develop a moisturizing lipstick, which shows good stability (i.e. anti-sweating performance).

Another object of the present invention is to provide a cosmetic process for caring for and/or making up keratin materials, which will deliver a moisturizing effect.

Thus, according to a first aspect, the present invention provides a solid anhydrous composition for caring for and/or making up keratin materials comprising,
a) 2 wt. % or more of at least one hydrophilic moisturizer, relative to the total weight of the composition;
b) at least one non-ionic surfactant selected from glyceryl fatty acid monoesters and polyglyceryl fatty acid esters;
c) polyethylene wax; and
d) at least one oil.

According to a second aspect, the present invention provides a cosmetic process for caring for and/or making up keratin materials comprising applying the solid anhydrous composition as described above to the keratin materials.

It has been found that with the combination of at least one non-ionic surfactant selected from glyceryl fatty acid monoesters, polyglyceryl fatty acid esters and polyethylene wax, the solid anhydrous composition according to the present invention do not have sweating issue for at least 1 month in 4° C., 25° C., 37° ° C., 45° C. ovens and in an oven undergoing a temperature variation cycle from −20° C. to 47° C. during one week.

It has been also found that the anti-sweating property in high humidity environment can be further improved in the presence of a thickener.

Other subjects and characteristics, aspects and advantages of the invention will emerge even more clearly on reading the description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

The expression “at least one” used in the present application is equivalent to the expression “one or more”.

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

For the purposes of the present invention, the term “anhydrous” means that the composition according to the present invention contains less than 2 wt. % and preferably less than 0.5 wt. % of water relative to the total weight of the composition. Where appropriate, such small amounts of water may be provided by ingredients of the composition that contain it in residual amount, but are not deliberately provided.

All percentages in the present application refer to weight percentage, unless otherwise specified.

As used herein, the term “keratin materials” refer to the skin and the lips. By “skin”, we intend to mean all the body skin, including the scalp. Preferably, the keratin material is the lips.

The solid anhydrous composition according to the present invention comprises:
a) 2 wt. % or more of at least one hydrophilic moisturizer, relative to the total weight of the composition;
b) at least one non-ionic surfactant selected from glyceryl fatty acid monoesters and polyglyceryl fatty acid esters;
c) polyethylene wax; and
d) at least one oil.

The term “solid” used herein means the hardness of the composition at 20° C. and at atmospheric pressure (760 mmHg) is greater than or equal to 30 Nm-1 when it is measured according to the protocol described below.

The composition whose hardness is to be determined is stored at 20° C. for 24 hours before measuring the hardness.

The hardness may be measured at 20° C. via the “cheese wire” method, which consists in transversely cutting a wand of product, which is preferably a circular cylinder, by means of a rigid tungsten wire 250 μm in diameter, by moving the wire relative to the stick at a speed of 100 mm/minute.

The hardness of the samples of compositions of the present invention, expressed in Nm-1, is measured using a DFGS2 tensile testing machine from the company Indelco-Chatillon.

The measurement is repeated three times and then averaged. The average of the three shear values read using the tensile testing machine mentioned above, noted Y, is given in grams. This average is converted into Newtons and then divided by L which represents the longest distance through which the wire passes. In the case of a cylindrical wand, L is equal to the diameter (in metres).

The hardness is converted into Nm 1 by the equation below:

$$( Y \times 1 {0}^{- 3} \times 9.8 ) / L$$

For a measurement at a different temperature, the composition is stored for 24 hours at this new temperature before the measurement.

According to this measuring method, the composition according to the present invention preferably has hardness at 20° C. and at atmospheric pressure of greater than or equal to 60 Nm⁻¹ and preferably greater than 75 Nm⁻¹.

Preferably, the composition according to the present invention especially has a hardness at 20° C. of less than 200 Nm⁻¹, and preferably less than 160 Nm⁻¹.

Advantageously, these compositions have a shear value ranging from 75 to 250 and preferably from 100 to 205 gF. Thus, these compositions may be formulated in standard packaging that does not require any composition support means.

### Hydrophilic Moisturizer(s)

According to the first aspect of the present invention, the solid anhydrous composition comprises 2 wt. % or more of hydrophilic moisturizer, relative to the total weight of the composition.

The moisturizers are dispersed in the lipophilic or continuous phase of the solid anhydrous composition of the present invention.

Said moisturizers are hydrophilic and comprise polyhydric alcohols, ethoxylated and propoxylated polyols, polysacharides, and mixtures thereof.

Preferred moisturizers are selected from the group consisting of glycerin, panthenol, hexylene glycol, polyethylene glycol, polypropylene glycol, sorbitol, and mixtures thereof. Most preferred is glycerin.

Advantageously, the hydrophilic moisturizer is present in the composition of the present invention in an amount ranging from 2 wt. % to 20 wt. %, preferably from 4 wt. % to 10 wt. %, relative to the total weight of the composition.

### Glyceryl Fatty Acid Monoesters and Polyglyceryl Fatty Acid Esters

According to the first aspect of the present invention, the solid anhydrous composition comprises at least one non-ionic surfactant selected from glyceryl fatty acid monoesters and polyglyceryl fatty acid esters.

The glyceryl fatty acid monoesters suitable for the present invention include, but are not limited to, glyceryl monoesters of C16-C22 saturated or unsaturated fatty acids.

Preferably, the glyceryl fatty acid monoester is selected from glyceryl oleate, glyceryl monostearate (or glyceryl stearate), glyceryl monoisostearate, glyceryl monopalmitate, glyceryl monobehenate, and mixtures thereof.

More preferably, the glyceryl fatty acid monoester used in the composition of the present invention is glyceryl monostearate (or glyceryl stearate).

The polyglyceryl fatty acid esters suitable for the present invention include, but are not limited to, polyglyceryl fatty acid esters with a polyglyceryl moiety derived from 2 to 10 glycerol units.

Preferably, the polyglyceryl fatty acid ester is selected from esters of one or more saturated or unsaturated fatty acid including 8 to 22, preferably 16 to 22 carbon atoms and polyglycerin with a polyglyceryl moiety derived from 2 to 10 glycerol units.

More preferably, the polyglycerol fatty acid ester is selected from polyglyceryl-4 caprate, polyglyceryl-2 caprate, polyglyceryl-4 caprylate, polyglyceryl-6 caprylate, polyglyceryl-6 caprate, polyglyceryl-4 caprylate/caprate, polyglyceryl-6 caprylate/caprate, polyglyceryl-3 cocoate, polyglyceryl-4 cocoate, polyglyceryl-10 decalinoleate, polyglyceryl-10 decaoleate, polyglyceryl-10 decacasterate, polyglyceryl-3 dicaprate, polyglyceryl-3 dicocoate, polyglyceryl-10 didecanoate, polyglyceryl-2 diisostearate, polyglyceryl-3 diisostearate, polyglyceryl-4 diisostearate,polyglyceryl-10 diisostearate, polyglyceryl-4 dilaurate, polyglycerin-2 dioleate, polyglyceryl-3 dioleate, polyglyceryl-6 dioleate, polyglyceryl-10 dioleate, polyglyceryl-6 dipalmitate, polyglyceryl-10 dipalmitate, polyglyceryl-2 dipolyhydroxystearate, polyglyceryl-2 distearate, polyglyceryl-3 distearate, polyglyceryl-6 distearate, polyglyceryl-10 distearate, polyglyceryl-10 heptaoleate, polyglyceryl-10 heptastearate, polyglyceryl-6 hexaoleate, polyglyceryl-10 hexaoleate, polyglyceryl-2 isopalmitate, polyglyceryl-2 isostearate, polyglyceryl-4 isostearate, polyglyceryl-4 diisostearate/polyhydroxystearate/sebacate, polyglyceryl-5 isostearate, polyglyceryl-6 isostearate, polyglyceryl-10 isostearate, polyglyceryl-2 laurate, polyglyceryl-3 laurate, polyglyceryl-4 laurate, polyglyceryl-4 laurate/sebacate, polyglyceryl-4 laurate/succinate, polyglyceryl-5 laurate, polyglyceryl-6 laurate, polyglyceryl-10 laurate, polyglyceryl-3 myristate, polyglyceryl-10 myristate, polyglyceryl-2 oleate, polyglyceryl-3 oleate, polyglyceryl-4 oleate, polyglyceryl-5 oleate, polyglyceryl-6 oleate, polyglyceryl-8 oleate, polyglyceryl-10 oleate, polyglyceryl-3 palmitate, polyglyceryl-6 palmitate, polyglyceryl-10 pentalaurate, polyglyceryl-10 pentalinoleate, polyglyceryl-4 pentaoleate, polyglyceryl-10 pentaoleate, polyglyceryl-3 pentaricinoleate, polyglyceryl-6 pentaricinoleate, polyglyceryl-10 pentaricinoleate, polyglyceryl-4 pentastearate, polyglyceryl-6 pentastearate, polyglyceryl-10 pentastearate, polyglyceryl-3 polyricinoleate, polyglyceryl-6 polyricinoleate, polyglyceryl-3 ricinoleate, polyglyceryl-3 stearate, polyglyceryl-2 stearate, polyglyceryl-4 stearate, polyglyceryl-8 stearate, polyglyceryl-10 stearate, polyglyceryl-2 tetraisostearate, polyglyceryl-6 tetraoleate, polyglyceryl-10 tetraoleate, polyglyceryl-2 tetrastearate, polyglyceryl-2 triisostearate, polyglyceryl-3 triisostearate, polyglyceryl-10 trioleate, polyglyceryl-4 tristearate, polyglyceryl-10 tristearate, and mixtures thereof.

Most preferably, the polyglyceryl fatty acid ester used in the composition of the present invention is polyglyceryl-6 polyricinoleate, polyglyceryl-4 diisostearate/polyhydroxystearate/sebacate, or mixtures thereof.

In some preferred embodiments, the non-ionic surfactant used is selected from glyceryl oleate, glyceryl monostearate, glyceryl monoisostearate, glyceryl monopalmitate, glyceryl monobehenate, polyglyceryl-3 pentaricinoleate, polyglyceryl-6 pentaricinoleate, polyglyceryl-10 pentaricinoleate, polyglyceryl-3 polyricinoleate, polyglyceryl-6 polyricinoleate, polyglyceryl-3 ricinoleate, polyglyceryl-2 isostearate, polyglyceryl-4 isostearate, polyglyceryl-5 isostearate, polyglyceryl-6 isostearate, polyglyceryl-10 isostearate, polyglyceryl-2 diisostearate, polyglyceryl-3 diisostearate, polyglyceryl-4 diisostearate, polyglyceryl-4 diisostearate/polyhydroxystearate/sebacate, polyglyceryl-10 diisostearate, polyglyceryl-2 tetraisostearate, polyglyceryl-2 triisostearate, polyglyceryl-3 triisostearate, and mixture thereof.

In some more preferred embodiments, the non-ionic surfactant used is selected from glyceryl monostearate, polyglyceryl-6 polyricinoleate, polyglyceryl-4 diisostearate/polyhydroxystearate/sebacate, polyglyceryl-4 isostearate, and mixture thereof.

Advantageously, the non-ionic surfactant selected from glyceryl fatty acid monoesters and polyglyceryl fatty acid esters is present in the composition of the present invention in an amount ranging from 0.1 wt. % to 30 wt. %, preferably from 1 wt. % to 8 wt. %, relative to the total weight of the composition.

### Polyethylene Wax

According to the first aspect of the present invention, the solid anhydrous composition comprises polyethylene wax.

Advantageously, polyethylene wax is present in the composition of the present invention in an amount ranging from 3 wt. % to 80 wt. %, preferably from 5 wt. % to 15 wt. %, relative to the total weight of the composition.

The inventors found that the presence of polyethylene wax is beneficial as far as the structural stability and lip sensation.

In addition to polyethylene wax, optionally, other wax(es) such as microcrystalline wax, synthetic wax, sunflower seed wax, candelilla wax and the like can be present.

### Oil(s)

According to the first aspect of the present invention, the solid anhydrous composition comprises at least one oil.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils, and fatty alcohols.

As examples of plant oils, mention may be made of, for example, canola oil, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, isostearate, linoleates, linolenates, caprates, and arachidonates, or mixtures thereof such as, especially, oleopalmitate, oleostearate, and palmitostearate mixed esters, as well as pentaerythrityl tetraethyl hexanoate, pentaerythrityl tetraisostearate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), pentaerythrityl tetraisostearate, 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) (or caprylic/capric triglyceride), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms; and
(ii) linear volatile polydialkylsiloxanes containing 2 to 9 silicon atoms and having a viscosity of less than or equal to 5×10⁻⁶ m²/s at 25° C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol is a saturated fatty alcohol.

Thus, the fatty alcohol may be selected from linear or branched, saturated or unsaturated C₆-C₃₀ alcohols, preferably linear or branched, saturated C₆-C₃₀ alcohols, and more preferably linear or branched, saturated C₁₂-C₂₀ alcohols.

The term “saturated fatty alcohol” here means an alcohol having a long aliphatic saturated carbon chain. It is preferable that the saturated fatty alcohol is selected from linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

In some preferred embodiments, the oil is selected from canola oil, squalane, pentaerythrityl tetraisostearate, caprylic/capric triglyceride, mixtures thereof.

Advantageously, the oil is present in the composition of the present invention in an amount ranging from 10 wt. % to 90 wt. %, preferably from 40 wt. % to 80 wt. %, relative to the total weight of the composition.

### Thickener(s)

Preferably, the composition according to the present invention comprises a thickener, in particular, a mineral thickener.

Mineral thickeners are mineral-based compounds that thicken or modify the viscosity of the cosmetic compositions.

Non-limiting examples of mineral thickener include silica silylate, fumed silica, zeolite, natural clay, synthetic clay, kaolin, hectorite, organically modified hectorite (e.g., INCI: pentaerythrityl tetraisostearate (and) disteardimonium hectorite (and) propylene carbonate), an activated clay (e.g., disteardimonium hectorite, stearalkonium hectorite, quaternium-18 bentonite, quaternium-18 hectorite, and benzalkonium bentonite), and a mixture thereof.

In some instance, the mineral thickening agents are chosen from silica silylate, fumed silica, zeolite, natural clay, synthetic clay, kaolin, hectorite, disteardimonium hectorite, stearalkonium hectorite, quaternium-18 bentonite, quaternium-18 hectorite, benzalkonium bentonite, and a mixture thereof.

Preferably, if presents, the total amount of the mineral thickener in the composition according to the present invention is 0.1 wt. % to 5 wt. %, based on the total weigh of the composition.

The total amount of the mineral thickener may be 0.2 wt. % to 3 wt. %, 0.2 wt. % to 3 wt. %, 0.5 wt. % to 1 wt. %, based on the total weight of the cosmetic composition.

The inventors found that the thickener can further improve the anti-sweating property of the composition according to the present invention under relatively higher humidity condition.

### Pasty Compound(s)

Optionally, the solid anhydrous composition comprises a pasty compound.

The term “pasty compound” used herein is understood to mean a lipophilic fatty compound with a reversible solid/liquid change of state exhibiting, in the solid state, an anisotropic crystalline arrangement and comprising, at a temperature of 23° C., a liquid fraction and a solid fraction.

In other words, the starting melting temperature of the pasty compound is less than 23° C. The liquid fraction of the pasty compound, measured at 23° C., represents 9 wt. % to 97 wt. % of the composition. This liquid fraction at 23° C. preferably represents between 15 wt. % and 85 wt. %, more preferably between 40 wt. % and 85 wt. %.

The melting point of a solid fatty substance can be measured using a differential scanning calorimeter (DSC), for example the calorimeter sold under the name “DSC Q100” by the company TA Instruments with the software “TA Universal Analysis”.

The measurement protocol is as follows.

A sample of 5 mg of pasty compound placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of pasty fatty substance is measured as a function of the temperature. The melting point of the pasty compound is the temperature value corresponding to the top of the peak of the curve representing the variation in the difference in power absorbed as a function of the temperature.

The liquid fraction by weight of the pasty compound at 23° C. is equal to the ratio of the heat of fusion consumed at 23° C. to the heat of fusion of the pasty compound.

The heat of fusion (expressed in J/g) of the pasty compound is the amount of energy required to make the compound change from the solid state to the liquid state. The pasty compound is said to be in the solid state when all of its mass is in crystalline solid form. The pasty compound is said to be in the liquid state when all of its mass is in liquid form. The heat of fusion consumed at 23° C. is the amount of energy absorbed by the sample to change from the solid state to the state that it has at 2° C., consisting of a liquid fraction and a solid fraction.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5° C. or 10° C. per minute, according to the standard ISO 11357-3; 1999.

The liquid fraction of the pasty compound measured at 32° C. preferably represents from 30% to 100% by weight of the pasty compound, preferably from 50% to 100% and more preferably from 60% to 100% by weight of the pasty compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same way as the heat of fusion consumed at 23° C.

The pasty compound(s) may in particular be chosen from synthetic pasty compounds and fatty substances of plant origin. The pasty compound(s) may be hydrocarbon-based or silicone-based.

The pasty compound(s) may be chosen in particular from:
lanolin and derivatives thereof, such as lanolin alcohol, oxyethylenated lanolins, acetylated lanolin, lanolin esters such as isopropyl lanolate, and oxypropylenated lanolins,
petroleum jelly (also known as petrolatum),
polyol ethers chosen from C₂-C₄ polyalkylene glycol pentaerythrityl ethers, fatty alcohol ethers of sugars, and mixtures thereof. For example, mention may be made of polyethylene glycol pentaerythrityl ether comprising 5 oxyethylene units (5 OE) (CTFA name: PPG-5 Pentaerythrityl Ether), polypropylene glycol pentaerythrityl ether comprising five oxypropylene (5 OP) units (CTFA name: PPG-5 Pentaerythrityl Ether) and mixtures thereof, and more especially the mixture PEG-5 Pentaerythrityl Ether, PPG-5 Pentaerythrityl Ether and soybean oil, sold under the name Lanolide by the company Vevy, which is a mixture in which the constituents are in a 46/46/8 weight ratio: 46% PEG-5 Pentaerythrityl Ether, 46% PPG-5 Pentaerythrityl Ether and 8% soybean oil,
polymeric or non-polymeric silicone compounds,
polymeric or non-polymeric fluoro compounds,
vinyl polymers, especially:
   olefin homopolymers and copolymers,
   hydrogenated diene homopolymers and copolymers,
   linear or branched oligomers, which are homopolymers or copolymers of alkyl (meth)acrylates preferably containing a C₈-C₃₀ alkyl group,
   oligomers, which are homopolymers and copolymers of vinyl esters containing C₈-C₃₀ alkyl groups, and
   oligomers, which are homopolymers and copolymers of vinyl esters containing C₈-C₃₀ alkyl groups,
liposoluble polyethers resulting from the polyetherification between one or more C₂-C₁₀₀ and preferably C₂-C₅₀ diols. Among the liposoluble polyethers that are particularly considered are copolymers of ethylene oxide and/or of propylene oxide with C₆-C₃₀ long-chain alkylene oxides, more preferably such that the weight ratio of the ethylene oxide and/or of the propylene oxide to the alkylene oxides in the copolymer is from 5:95 to 70:30. In this family, mention will be made especially of copolymers such as long-chain alkylene oxides arranged in blocks with an average molecular weight from 1000 to 10 000, for example a polyoxyethylene/polydodecyl glycol block copolymer such as the ethers of dodecanediol (22 mol) and of polyethylene glycol (45 OE) sold under the brand name Elfacos ST9 by Akzo Nobel,
esters and polyesters. Among the esters, the following are especially considered:
   esters of a glycerol oligomer, especially diglycerol esters, with linear or branched, saturated or unsaturated, preferably saturated, C₆-C₂₀, optionally hydroxylated monocarboxylic acids, and/or linear or branched, saturated or unsaturated, preferably saturated, C₆-C₁₀ dicarboxylic acids, in particular condensates of adipic acid and of diglycerol, for which some of the hydroxyl groups of the glycerols have reacted with a mixture of fatty acids such as stearic acid, capric acid, isostearic acid and 12-hydroxystearic acid, for instance bis-diglyceryl polyacyladipate-2 sold under the reference Softisan® 649 by the company Sasol,
   vinyl ester homopolymers bearing C₈-C₃₀ alkyl groups, such as polyvinyl laurate (sold especially under the reference Mexomer PP by the company Chimex),
   the arachidyl propionate sold under the brand name Waxenol 801 by Alzo,
   phytosterol esters,
   fatty acid triglycerides and derivatives thereof, in particular optionally hydrogenated (totally or partially), optionally monohydroxylated or polyhydroxylated, C₆-C₃₀ and more particularly C₈-C₁₈, linear or branched, saturated or unsaturated fatty acid triglycerides; for example Softisan 100® sold by the company Sasol,
   pentaerythritol esters,
   aliphatic esters resulting from the esterification of an aliphatic hydroxycarboxylic acid ester with an aliphatic carboxylic acid. More particularly, the aliphatic carboxylic acid is of C₄-C₃₀ and preferably of C₈-C₃₀. It is preferably chosen from hexanoic, heptanoic, octanoic, 2-ethylhexanoic, decanoic, undecanoic, dodecanoic, tridecanoic, tetradecanoic, pentadecanoic, hexadecenoic, hexyldecanoic, heptadecanoic, octadecanoic, isostearic, nonadecanoic, eicosanoic, isoarachidic, octyldodecanoic, heneicosanoic and docosanoic acids, and mixtures thereof. The aliphatic carboxylic acid is preferably branched. The hydroxy carboxylic acid ester is advantageously derived from a C₂-C₄₀, preferably C₁₀-C₃₄ and even more preferentially C₁₂-C₂₈ hydroxylated carboxylic acid; the number of hydroxyl groups being between 1 and 20, more particularly between 1 and 10 and preferably between 1 and 6.
   esters of a diol dimer and of a diacid dimer, where appropriate esterified on their free alcohol or acid function(s) with acid or alcohol radicals, especially dimer dilinoleate esters; such esters may be chosen especially from the esters having the following INCI nomenclature: bis-behenyl/isostearyl/phytosteryl dimer dilinoleyl dimer dilinoleate (Plandool G), phytosteryl/isosteryl/cetyl/stearyl/behenyl dimer dilinoleate (Plandool H or Plandool S), and mixtures thereof,
   hydrogenated esters of rosin (Lusplan DD-DHR or DD-DHR from Nippon Fine Chemical);
butters of plant origin, such as mango butter, such as the product sold under the reference Lipex 203 by the company Aarhuskarlshamn, shea butter, in particular the product whose INCI name is Butyrospermum Parkii Butter, such as the product sold under the reference Sheasoft® by the company Aarhuskarlshamn, cupuacu butter (Rain Forest RF3410 from the company Beraca Sabara), murumuru butter (Rain Forest RF3710 from the company Beraca Sabara), cocoa butter; babassu butter such as the product sold under the name Cropure Babassu SS-(LK) by Croda, and also orange wax, for instance the product sold under the reference Orange Peel Wax by the company Koster Keunen,
totally or partially hydrogenated plant oils, for instance hydrogenated soybean oil, hydrogenated coconut oil, hydrogenated rapeseed oil, mixtures of hydrogenated plant oils such as the mixture of hydrogenated soybean, coconut, palm and rapeseed plant oil, for example the mixture sold under the reference Akogel® by the company Aarhuskarlshamn (INCI name Hydrogenated Vegetable Oil), the trans-isomerized partially hydrogenated jojoba oil manufactured or sold by the company Desert Whale under the commercial reference Iso-Jojoba-500, partially hydrogenated olive oil, for instance the compound sold under the reference Beurrolive by the company Soliance,
hydrogenated castor oil esters, such as hydrogenated castor oil dimer dilinoleate, for example Risocast DA-L sold by Kokyu Alcohol Kogyo, and hydrogenated castor oil isostearate, for example Salacos HCIS (V-L) sold by Nisshin Oil,
and mixtures thereof.

Preferably, the pasty compound(s) used in the invention are chosen from esters of a diol dimer and of a diacid dimer, for instance dilinoleate dimer esters.

More preferably, the pasty compound is selected from the compound of formula (I):

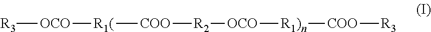

in which:
COR₁CO represents a dimer dilinoleate residue,
OR₂O represents a fatty alcohol dimer residue having from 16 to 68, from 24 to 44, in particular from 32 to 40, and more particularly 36 carbon atoms,
OR₃ represents a monoalcohol residue having from 4 to 40, in particular from 6 to 36, in particular from 8 to 32, in particular from 16 to 28, and more particularly from 18 to 24 carbon atoms, and
n is an integer ranging from 1 to 15, in particular from 2 to 10 and more particularly from 5 to 7.

Most preferably, the pasty compound is selected from the group consisting of esters having the following INCI nomenclature: polyglyceryl-2 isostearate dimer dilinoleate copolymer, bis-behenyl/isostearyl/phytosteryl dimer dilinoleyl dimer dilinoleate, and mixtures thereof.

Preferably, if presents, the pasty compound is present in the composition of the present invention in an amount ranging from 0.01 wt. % to 60 wt. %, preferably from 0.01 wt. % to 30 wt. %, relative to the total weight of the composition.

### Colorant(s)

The solid anhydrous composition according to the present invention may optionally comprise at least one colorant. Preferably, the amount of colorant(s) is below 20 wt. % relative to the total weight of the composition.

For the purposes of the present invention, the term “colorant” means a compound that is capable of producing a colored optical effect when it is formulated in sufficient amount in a suitable cosmetic medium.

The colorant under consideration in the context of the present invention may be selected from water-soluble or water-insoluble, liposoluble or non-liposoluble, organic or inorganic colorants, and materials with an optical effect, and mixtures thereof.

### Water-Soluble Dyes

The water-soluble colorants used according to the present invention are more particularly water-soluble dyes.

For the purposes of the present invention, the term “water-soluble dye” means any natural or synthetic, generally organic compound which is soluble in an aqueous phase or water-miscible solvents and which is capable of imparting colour. In particular, the term “water-soluble” is intended to characterize the capacity of a compound to dissolve in water, measured at 25° C., to a concentration at least equal to 0.1 g/l (production of a macroscopically isotropic, transparent, coloured or colourless solution). This solubility is in particular greater than or equal to 1 g/l.

As water-soluble dyes that are suitable for use in the present invention, mention may be made in particular of synthetic or natural water-soluble dyes, for instance FD&C Red 4 (CI: 14700), DC Red 6 (Lithol Rubine Na; CI: 15850), DC Red 22 (CI: 45380), DC Red 28 (CI: 45410 Na salt), DC Red 30 (CI: 73360), DC Red 33 (CI: 17200), DC Orange 4 (CI: 15510), FDC Yellow 5 (CI: 19140), FDC Yellow 6 (CI: 15985), DC Yellow 8 (CI: 45350 Na salt), FDC Green 3 (CI: 42053), DC Green 5 (CI: 61570), FDC Blue 1 (CI: 42090).

As non-limiting illustrations of sources of water-soluble colorant(s) that may be used in the context of the present invention, mention may be made in particular of those of natural origin, such as extracts of cochineal carmine, of beetroot, of grape, of carrot, of tomato, of annatto, of paprika, of henna, of caramel and of curcumin.

Thus, the water-soluble colorants that are suitable for use in the present invention are in particular carminic acid, betanin, anthocyans, enocyanins, lycopene, β-carotene, bixin, norbixin, capsanthin, capsorubin, flavoxanthin, lutein, cryptoxanthin, rubixanthin, violaxanthin, riboflavin, rhodoxanthin, cantaxanthin and chlorophyll, and mixtures thereof.

They may also be copper sulfate, iron sulfate, water-soluble sulfopolyesters, rhodamine, betaine, methylene blue, the disodium salt of tartrazine and the disodium salt of fuchsin.

Some of these water-soluble colorants are in particular approved for food use. Representatives of these dyes that may be mentioned more particularly include dyes of the carotenoid family, referenced under the food codes E120, E162, E163, E160a-g, E150a, E101, E100, E140 and E141.

### Pigments

The term “pigments” should be understood as meaning white or coloured, inorganic (mineral) or organic particles, which are insoluble in a liquid organic phase, and which are intended to color and/or opacify the composition and/or the deposit produced with the composition.

The pigments may be selected from mineral pigments, organic pigments and composite pigments (i.e. pigments based on mineral and/or organic materials).

The pigments may be selected from monochromatic pigments, lakes and pigments with an optical effect, for instance goniochromatic pigments and nacres.

The mineral pigments may be selected from metal oxide pigments such as chromium oxides, iron oxides (black, yellow, red), titanium dioxide, zinc oxides, cerium oxides and, zirconium oxides, chromium hydrate, manganese violet, Prussian blue, ultramarine blue, ferric blue, SYNTHETIC FLUORPHLOGOPITE, metal powders such as aluminium powders and copper powder, and mixtures thereof.

Organic lakes are organic pigments formed from a dye attached to a substrate.

The lakes, which are also known as organic pigments, may be selected from the materials below, and mixtures thereof:
cochineal carmine;
organic pigments of azo dyes, anthraquinone dyes, indigoid dyes, xanthene dyes, pyrene dyes, quinoline dyes, triphenylmethane dyes or fluorane dyes.

Among the organic pigments that may in particular be mentioned are those known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6;
the organic lakes may be insoluble sodium, potassium, calcium, barium, aluminium, zirconium, strontium or titanium salts of acidic dyes such as azo, anthraquinone, indigoid, xanthene, pyrene, quinoline, triphenylmethane or fluorane dyes, these dyes possibly comprising at least one carboxylic or sulfonic acid group.

The organic lakes may also be supported on an organic support such as rosin or aluminium benzoate, for example.

Among the organic lakes, mention may be made in particular of those known under the following names: D&C Red No. 2 Aluminium lake, D&C Red No. 3 Aluminium lake, D&C Red No. 4 Aluminium lake, D&C Red No. 6 Aluminium lake, D&C Red No. 6 Barium lake, D&C Red No. 6 Barium/Strontium lake, D&C Red No. 6 Strontium lake, D&C Red No. 6 Potassium lake, D&C Red No. 7 Aluminium lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminium lake, D&C Red No. 9 Barium lake, D&C Red No. 9 Barium/Strontium lake, D&C Red No. 9 Zirconium lake, D&C Red No. 10 Sodium lake, D&C Red No. 19 Aluminium lake, D&C Red No. 19 Barium lake, D&C Red No. 19 Zirconium lake, D&C Red No. 21 Aluminium lake, D&C Red No. 21 Zirconium lake, D&C Red No. 22 Aluminium lake, D&C Red No. 27 Aluminium lake, D&C Red No. 27 Aluminium/Titanium/Zirconium lake, D&C Red No. 27 Barium lake, D&C Red No. 27 Calcium lake, D&C Red No. 27 Zirconium lake, D&C Red No. 28 Aluminium lake, D&C Red No. 30 lake, D&C Red No. 31 Calcium lake, D&C Red No. 33 Aluminium lake, D&C Red No. 34 Calcium lake, D&C Red No. 36 lake, D&C Red No. 40 Aluminium lake, D&C Blue No. 1 Aluminium lake, D&C Green No. 3 Aluminium lake, D&C Orange No. 4 Aluminium lake, D&C Orange No. 5 Aluminium lake, D&C Orange No. 5 Zirconium lake, D&C Orange No. 10 Aluminium lake, D&C Orange No. 17 Barium lake, D&C Yellow No. 5 Aluminium lake, D&C Yellow No. 5 Zirconium lake, D&C Yellow No. 6 Aluminium lake, D&C Yellow No. 7 Zirconium lake, D&C Yellow No. 10 Aluminium lake, FD&C Blue No. 1 Aluminium lake, FD&C Red No. 4 Aluminium lake, FD&C Red No. 40 Aluminium lake, FD&C Yellow No. 5 Aluminium lake and FD&C Yellow No. 6 Aluminium lake.

Mention may also be made of liposoluble dyes, such as, for example, Sudan Red, DC Red 17, DC Green 6, B-carotene, soybean oil, Sudan Brown, DC Yellow 11, DC Violet 2, DC Orange 5 and quinoline yellow.

The chemical substances corresponding to each of the organic colorants cited above are mentioned in the publication “International Cosmetic Ingredient Dictionary and Handbook”, 1997 edition, pages 371 to 386 and 524 to 528, published by “The Cosmetic, Toiletries and Fragrance Association”, the content of which is incorporated into the present patent application by way of reference.

The pigments may also have been subjected to a hydrophobic treatment.

The hydrophobic treatment agent may be selected from silicones such as methicones, dimethicones, alkoxysilanes (e.g., triethoxysilylethyl polydimethylsiloxyethyl dimethicone) and perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, polyhexafluoropropylene oxides, polyorganosiloxanes comprising perfluoroalkyl perfluoropolyether groups, and amino acids; N-acylamino acids or salts thereof; lecithin, isopropyl triisostearyl titanate, and mixtures thereof.

The N-acylamino acids can comprise an acyl group containing from 8 to 22 carbon atoms, such as, for example, a 2-ethylhexanoyl, caproyl, lauroyl, myristoyl, palmitoyl, stearoyl or cocoyl group. The salts of these compounds may be aluminium, magnesium, calcium, zirconium, zinc, sodium or potassium salts. The amino acid may be, for example, lysine, glutamic acid or alanine.

The term “alkyl” mentioned in the compounds cited above in particular denotes an alkyl group containing from 1 to 30 carbon atoms and preferably containing from 5 to 16 carbon atoms.

Hydrophobically treated pigments are described in particular in patent application EP-A-1 086 683.

### Nacres

For the purposes of the present patent application, the term “nacre” means coloured particles of any shape, which may or may not be iridescent, in particular produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, titanium mica coated with an organic dye in particular of the abovementioned type, and also nacreous pigments based on bismuth oxychloride.

They may also be mica particles, at the surface of which are superposed at least two successive layers of metal oxides and/or of organic colorants.

The nacres may more particularly have a yellow, pink, red, bronze, orangey, brown, gold and/or coppery colour or tint.

As illustrations of nacres that may be introduced as interference pigments into the first composition, mention may be made of the gold-coloured nacres sold in particular by the company BASF under the name Brilliant gold 212G (Timica), Gold 222C (Cloisonne), Sparkle gold (Timica) and Monarch gold 233X (Cloisonne); the bronze nacres sold in particular by the company Merck under the name Bronze fine (17384) (Colorona) and Bronze (17353) (Colorona) and by the company BASF under the name Super bronze (Cloisonne); the orange nacres sold in particular by the company BASF under the name Orange 363C (Cloisonne) and by the company Merck under the name Passion orange (Colorona) and Matte orange (17449) (Microna); the brown tinted nacres sold in particular by the company Engelhard under the name Nu-antique copper 340XB (Cloisonne) and Brown CL4509 (Chroma-lite); the copper-tinted nacres sold in particular by the company BASF under the name Copper 340A (Timica); the red-tinted nacres sold in particular by the company Merck under the name Sienna fine (17386) (Colorona); the yellow-tinted nacres sold in particular by the company BASF under the name Yellow (4502) (Chromalite); the gold-tinted red nacres sold in particular by the company BASF under the name Sunstone G012 (Gemtone); the pink nacres sold in particular by the company BASF under the name Tan opal G005 (Gemtone); the gold-tinted black nacres sold in particular by the company BASF under the name Nu antique bronze 240 AB (Timica), the blue nacres sold in particular by the company Merck under the name Matte blue (17433) (Microna), the silvery-tinted white nacres sold in particular by the company Merck under the name Xirona Silver, and the golden-green pink-orange nacres sold in particular by the company Merck under the name Indian summer (Xirona), and mixtures thereof.

### Goniochromatic Pigments

For the purposes of the present invention, the term “goniochromatic pigment” denotes a pigment which makes it possible to obtain, when the composition is spread onto a support, a colour trajectory in the a*b* plane of the CIE 1976 colorimetric space that corresponds to a variation Dh in the hue angle ho of at least 20° when the angle of observation relative to the normal is varied between 0° and 80°, for an incident light angle of 45°.

The color trajectory may be measured, for example, using an Instrument Systems brand spectrogonioreflectometer of reference GON 360 Goniometer, after the composition has been spread in fluid form to a thickness of 300 μm using an automatic spreader onto an Erichsen brand contrast card of reference Typ 24/5, the measurement being taken on the black background of the card.

The goniochromatic pigment may be chosen, for example, from multilayer interference structures and liquid-crystal colouring agents.

In the case of a multilayer structure, it may comprise, for example, at least two layers, each layer being made, for example, from at least one material selected from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and combinations thereof.

The multilayer structure may or may not have, relative to a central layer, symmetry in the chemical nature of the stacked layers.

Different effects are obtained depending on the thickness and the nature of the various layers.

Examples of symmetrical multilayer interference structures are, for example, the following structures: Fe₂O₃/SiO₂/Fe₂O₃/SiO₂/Fe₂O₃, a pigment having this structure being sold under the name Sicopearl by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the name Xirona by the company Merck.

The liquid-crystal coloring agents comprise, for example, silicones or cellulose ethers onto which are grafted mesomorphic groups. Examples of liquid-crystal goniochromatic particles that may be used include, for example, those sold by the company Chenix and also those sold under the name Helicone HC by the company Wacker.

Goniochromatic pigments that may also be used include certain nacres, pigments with effects on a synthetic substrate, in particular a substrate such as alumina, silica, borosilicate, iron oxide or aluminium, or interference flakes obtained from a polyterephthalate film.

By way of nonlimiting examples of goniochromatic pigments, mention may in particular be made, alone or in mixtures, of SunShine® goniochromatic pigments sold by SunChemicals, Cosmicolor Celeste @ from Toyo Aluminium K.K., Xirona @ from Merck and Reflecks Multidimensions® from BASF.

Optionally, these particles may comprise or be covered with optical brightener(s) (or organic white fluorescent substances).

Optical brighteners are compounds well known to a person skilled in the art. Such compounds are described in “Fluorescent Whitening Agent, Encyclopedia of Chemical Technology, Kirk-Othmer”, vol. 11, pp. 227-241, 4th Edition, 1994, Wiley.

Their use in cosmetics in particular exploits the fact that they consist of chemical compounds having fluorescence properties, which absorb in the ultraviolet region (maximum absorption at a wavelength of less than 400 nm) and re-emit energy by fluorescence for a wavelength of between 380 nm and 830 nm. They may be defined more particularly as compounds that absorb essentially in the UVA region between 300 and 390 nm and re-emit essentially between 400 and 525 nm. Their lightening effect is based more particularly on an emission of energy between 400 and 480 nm, which corresponds to an emission in the blue part of the visible region, which contributes to lightening the skin visually when this emission takes place on the skin.

Optical brighteners that are in particular known include stilbene derivatives, in particular polystyrylstilbenes and triazinylstilbenes, coumarin derivatives, in particular hydroxycoumarins and aminocoumarins, oxazole, benzoxazole, imidazole, triazole and pyrazoline derivatives, pyrene derivatives, porphyrin derivatives and mixtures thereof.

The optical brighteners that can be used may also be in the form of copolymers, for example of acrylates and/or methacrylates, grafted with optical brightener groups as described in application FR 99 10942.

According to a preferred embodiment, the colorant used in the present invention is selected from metal oxide pigments, organic lakes, synthetic or natural water-soluble dyes and mixtures thereof.

According to a particularly preferred embodiment, the colorant used in the present invention is selected from YELLOW 6 LAKE, Blue 1 lake, RED 28 LAKE, RED 21, RED 7, titanium dioxide, iron oxides, synthetic fluorphlogopite, and mixtures thereof.

Preferably, if presents, the colorant is present in an amount ranging from 0.01 wt. % to 35 wt. %, preferably from 5 wt. % to 15 wt. %, relative to the total weight of the composition.

### Other Ingredients

The solid anhydrous composition according to the present invention may further comprise other ingredient(s) usually used in the field under consideration.

For example, the solid anhydrous composition according to the present invention may further comprises other ingredients selected from polymers for wear improvement, fillers, antioxidants, preserving agents, fragrances, neutralizers, antiseptics, additional cosmetic active agents, such as vitamins, collagen-protecting agents, and mixtures thereof.

It was found that the solid anhydrous composition according to the present invention can demonstrate an improved anti-sweating performance in the presence of a thickener.

It is a matter of routine operations for a person skilled in the art to adjust the nature and amount of the other ingredients present in the compositions in accordance with the present invention such that the advantageous properties of the composition used according to the present invention are not, or are not substantially, adversely affected by the envisaged addition.

According to a preferred embodiment, the present invention provides a solid anhydrous composition comprising, relative to the total weight of the composition:
a) from 4 wt. % to 10 wt. % of glycerin;
b) from 1 wt. % to 8 wt. % of at least one non-ionic surfactant selected from glyceryl monostearate, polyglyceryl-6 polyricinoleate, polyglyceryl-4 diisostearate/polyhydroxystearate/sebacate, polyglyceryl-4 isostearate, and mixture thereof;
c) from 5 wt. % to 15 wt. % of polyethylene wax;
d) from 40 wt. % to 80 wt. % at least one oil selected from canola oil, squalane, pentaerythrityl tetraisostearate, caprylic/capric triglyceride, mixtures thereof; and
e) from 5 wt. % to 15 wt. % of a colorant selected from YELLOW 6 LAKE, Blue 1 lake, RED 28 LAKE, RED 21, RED 7, titanium dioxide, iron oxides, synthetic fluorphlogopite, and mixtures thereof.

### Galenic Form

The solid anhydrous composition of the present invention is suitable to be used as a skin care, and/or make up product. More particularly, the composition of the present invention is in the form of lipstick and so on.

The composition according to the present invention may be prepared in a conventional manner.

According to the second aspect, the present invention provides a cosmetic process for caring for/making up keratin materials comprising applying the solid anhydrous composition as described above to the keratin materials.

The examples that follow are given as non-limiting illustrations of the present invention.

### EXAMPLES

Main raw materials used, trade names and supplier thereof are listed in Table 1.

TABLE 1

| INCI Name | Trade Name | Supplier |
|---|---|---|
| CANOLA OIL | LIPEX PREACT ™ | AARHUSKARLSHAMN |
| SQUALANE | NEOSSANCE ™ SQUALANE | AMYRIS |
| PENTAERYTHRITYL | JOLEE 7181 | OLEON |
| TETRAISOSTEARATE | | |
| CAPRYLIC/CAPRIC | MASESTER E7000 | PT MUSIM MAS |
| TRIGLYCERIDE | | |
| TOCOPHEROL | NOVATOL ™ 5-87 | ADM |
| POLYGLYCERYL-6 | SY-GLYSTER CRS-75 | SAKAMOTO YAKUHIN |
| POLYRICINOLEATE | | |
| GLYCERYL STEARATE | TEGIN ® 90 PELLETS | EVONIK |
| | | GOLDSCHMIDT |
| POLYGLYCERYL-4 | ISOLAN ® GPS | EVONIK |
| DIISOSTEARATE/POLYHYDROXY | | GOLDSCHMIDT |
| STEARATE/SEBACATE | | |
| POLYGLYCERYL-4 ISOSTEARATE | ISOLAN GI 34 | EVONIK |
| | | GOLDSCHMIDT |
| GLYCERIN | CREMERGLYC REFINED | CREMER OLEO (IOI) |
| | GLYCERINE 99.7% RSPO MB | |
| BISBEHENYL/ISOSTEARYL/ | Plandool ™ G | NIPPON FINE |
| PHYTOSTERYL | | CHEMICAL |
| DIMER DILINOLEYL | | |
| DIMER DILINOLEATE | | |
| POLYETHYLENE | PERFORMALENE ™ 500 | NEW PHASE |
| | POLYETHYLENE | TECHNOLOGIES |
| HELIANTHUS ANNUUS | SUNFLOWER WAX DOUBLE | KOSTER KEUNEN |
| (SUNFLOWER) SEED WAX | REFINED | |
| EUPHORBIA CERIFERA | 7820 LIGHT SPECIAL CANDELILLA | MULTICERAS |
| (CANDELILLA) WAX | REAL ® | |
| ORYZA SATIVA (RICE) BRAN | NC 1720 | CERA RICA NODA |
| WAX | | |
| RED 7 | UNIPURE RED LC 3079 OR | SENSIENT |
| YELLOW 6 LAKE | SUNCROMA ™ FD&C YELLOW 6 AL | SUN |
| | LAKE C70-5270 | |
| RED 28 LAKE | SUNCROMA ™ D&C RED 28 AL | SUN |
| | LAKE C14-6623 | |
| DISTEARDIMONIUM | BENTONE ® 38 | ELEMENTIS |
| HECTORITE | | |

### Invention Examples 1-6 and Comparative Examples 1-4

Lipsticks according to the present invention (IE.) 1-6 and comparative lipstick (CE) 1-4 were prepared with the ingredients listed in Tables 2 and 3 (the contents are expressed as weight percentages of active material, unless otherwise indicated): 5

TABLE 2

| Components | Ex. 1 | CE. 1 | CE. 2 | CE. 3 | CE. 4 |
|---|---|---|---|---|---|
| CANOLA OIL | QS 100 | | | | |
| SQUALANE | 12.50 | 12.50 | 12.50 | 12.50 | 12.50 |
| PENTAERYTHRITYL | 10 | 10 | 10 | 10 | 10 |
| TETRAISOSTEARATE | | | | | |
| CAPRYLIC/CAPRIC | 5.37 | 5.37 | 5.37 | 5.37 | 5.37 |
| TRIGLYCERIDE | | | | | |
| TOCOPHEROL | 0.80 | 0.80 | 0.80 | 0.80 | 0.80 |
| POLYGLYCERYL-6 | 3 | — | 3 | 3 | 3 |
| POLYRICINOLEATE | | | | | |
| GLYCERIN | 8 | 8 | 8 | 8 | 8 |
| BIS-BEHENYL/ISOSTEARYL/ | 8.5 | 8.5 | 8.5 | 8.5 | 8.5 |
| PHYTOSTERYL DIMER | | | | | |
| DILINOLEYL DIMER | | | | | |
| DILINOLEATE | | | | | |
| POLYETHYLENE | 11 | 11 | — | — | — |
| HELIANTHUS ANNUUS | — | — | 11 | 6.73 | 3.14 |
| (SUNFLOWER) SEED WAX | | | | | |
| EUPHORBIA CERIFERA | — | — | — | 4.27 | 3.14 |
| (CANDELILLA) WAX | | | | | |
| ORYZA SATIVA | — | — | — | — | 4.72 |
| (RICE) BRAN WAX | | | | | |
| RED 7 | 2.79 | 2.79 | 2.79 | 2.79 | 2.79 |
| YELLOW 6 LAKE | 5.65 | 5.65 | 5.65 | 5.65 | 5.65 |
| RED 28 LAKE | 1.56 | 1.56 | 1.56 | 1.56 | 1.56 |

TABLE 3

| Components | Ex. 2 | Ex. 3 | Ex. 4 | Ex. 5 | Ex. 6 |
|---|---|---|---|---|---|
| CANOLA OIL | QS 100 | | | | |
| SQUALANE | 12.50 | 12.50 | 12.50 | 12.50 | 12.50 |
| PENTAERYTHRITYL | 10 | 10 | 10 | 10 | 10 |
| TETRAISOSTEARATE | | | | | |
| CAPRYLIC/CAPRIC | 5.37 | 5.37 | 5.37 | 5.37 | 5.37 |
| TRIGLYCERIDE | | | | | |
| TOCOPHEROL | 0.80 | 0.80 | 0.80 | 0.80 | 0.80 |
| POLYGLYCERYL-6 | — | — | 3.75 | 5 | — |
| POLYRICINOLEATE | | | | | |
| POLYGLYCERYL-4 | — | — | 1.25 | — | — |
| ISOSTEARATE | | | | | |
| GLYCERYL STEARATE | 3 | — | — | — | — |
| POLYGLYCERYL-4 | — | 5 | — | — | 5 |
| DIISOSTEARATE/ | | | | | |
| POLYHYDROXYSTEARATE/ | | | | | |
| SEBACATE | | | | | |
| DISTEARDIMONIUM | — | — | — | 0.8 | 0.8 |
| HECTORITE | | | | | |
| GLYCERIN | 8 | 8 | 8 | 8 | 8 |
| BIS-BEHENYL/ISOSTEARYL/ | 8.5 | 8.5 | 8.5 | 8.5 | 8.5 |
| PHYTOSTERYL DIMER | | | | | |
| DILINOLEYL DIMER | | | | | |
| DILINOLEATE | | | | | |
| POLYETHYLENE | 11 | 11 | 11 | 11 | 11 |
| RED 7 | 2.79 | 2.79 | 2.79 | 2.79 | 2.79 |
| YELLOW 6 LAKE | 5.65 | 5.65 | 5.65 | 5.65 | 5.65 |
| RED 28 LAKE | 1.56 | 1.56 | 1.56 | 1.56 | 1.56 |

### Preparation Procedure:

The detailed procedure of preparing above lipsticks is as below:
1). Pigments (Red 7, Red 28 Lake, and Yellow 6 Lake) were ground with canola oil using a three-roll mill;
2). Oils, waxes, surfactants and pasty compounds were weighed into a main vessel and heat the vessel to around 95° C. with stirring until a homogeneous mixture was obtained;
3). Add glycerin into the main vessel and homogenize the bulk at a strong agitation speed;
4). Add the pigment paste prepared in step 1);
5). Pouring the homogenized mixture into a lipstick mold preheated to 42+2° C., leaving the mixture in the mold under 25° C. until solidation; and
6) Demolding the lipsticks from the lipstick mold.

### Evaluation:

Evaluation on the shear value, hardness, and anti-sweating property of the lipsticks prepared were performed.

The shear value and hardness was evaluated according to the protocol described previously.

The shear value and hardness results of each lipstick were summarized in Table 4.

TABLE 4

| | Shear value (gF) | Hardness (Nm⁻¹)* |
|---|---|---|
| EX. 1 | 138 | 106 |
| CE. 1 | 135 | 104 |
| CE. 2 | 125 | 96 |
| CE. 3 | 108 | 83 |
| CE. 4 | 120 | 93 |
| EX. 2 | 202 | 156 |
| EX. 3 | 140 | 108 |
| EX. 4 | 138 | 106 |
| EX. 5 | 135 | 104 |
| EX. 6 | 144 | 111 |

*Calculated based on that the diameter L of the lipstick is 12.7 mm.

The anti-sweating property of the lipsticks was evaluated as follows.

Firstly, lipsticks were put in the oven with constant temperature of 20° C.

After 24 hours, lipsticks were put in 6 tested ovens:
one Cycle oven: undergoing a temperature variation cycle of about one week from −20° ° C. to 47° C.;
one HUM oven: set to 37° C. and 80% RH (relative humidity); and
4 ovens: set to 4° C., 25° C., 37° C. and 45° C. respectively without humidity control.

Generally, 2 samples for each lipstick (one stands up and the other upside down) were put in each oven at the same time, except in the HUM oven where one sample stands up without cap). At each checkpoint, lipsticks were taken out, checked the appearance of each lipstick, recorded the data and put them back.

The anti-sweating results of lipsticks were summarized in Tables 5 and 6, wherein Y means sweating (a number of droplets seen on the lipstick surface); N means no sweating.

TABLE 5

| | | | | |
|---|---|---|---|---|
| IE. 1 | CE. 1 | CE. 2 | CE. 3 | CE. 4 |

At T = 1 hours

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | N | Y | Y | Y | Y |

At T = 4 hours

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | N | Y | Y | Y | Y |

At T = 24 hours

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | Y | Y | Y | Y | Y |

At T = 3 days

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | Y | Y | Y | Y | Y |

At T = 1 week

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | Y | Y | Y | Y | Y |
| Cycle | N | Y | Y | Y | Y |

At T = 1 month

| | | | | | |
|---|---|---|---|---|---|
| 4° C. | N | Y | N | N | N |
| 25° C. | N | Y | N | N | N |
| 37° C. | N | Y | Y | Y | Y |
| 45° C. | N | Y | Y | Y | Y |
| HUM | Y | Y | Y | Y | Y |

TABLE 6

| | | | | |
|---|---|---|---|---|
| IE. 2 | IE. 3 | IE. 4 | IE. 5 | IE. 6 |

At T = 1 hour

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | N | N | N | N | N |

At T = 4 hours

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | N | N | N | N | N |

At T = 24 hours

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | N | N | N | N | N |

At T = 3 days

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | N | Y | N | N | N |

At T = 1 week

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | N | Y | N | N | N |
| Cycle | N | N | N | N | N |

At T = 1 month

| | | | | | |
|---|---|---|---|---|---|
| 4° C. | N | N | N | N | N |
| 25° C. | N | N | N | N | N |
| 37° C. | N | N | N | N | N |
| 45° C. | N | N | N | N | N |
| HUM | Y | Y | N | N | N |

It can be seen from Tables 5 and 6 that the solid anhydrous composition according to the present invention do not have sweating issue for at least 1 month in ovens at 4° C., 25° C., 37° C., or 45° C. and in an oven undergoing a temperature variation cycle from −20° ° C. to 47° C. during one week.

It can also be seen that the anti-sweating property under relatively higher humidity environment can be further improved by the presence of a thickener.

1. A solid anhydrous composition for caring for and/or making up keratin materials comprising,
a) 2 wt. % or more of at least one hydrophilic moisturizer, relative to the total weight of the composition,
b) at least one non-ionic surfactant selected from glyceryl fatty acid monoesters and polyglyceryl fatty acid esters,
c) polyethylene wax, and
d) at least one oil.

2. The composition according to claim 1, wherein the hydrophilic moisturizer is selected from the group consisting of glycerin, panthenol, hexylene glycol, polyethylene glycol, polypropylene glycol, sorbitol, and mixtures thereof.

3. The composition according to claim 1, wherein the hydrophilic moisturizer is present in an amount ranging from 2 wt. % to 20 wt. % relative to the total weight of the composition.

4. The composition according to claim 1, wherein the non-ionic surfactant is selected from the group consisting of glyceryl monoesters of one or more C16-C22 saturated or unsaturated fatty acids, and esters of saturated or unsaturated fatty acid including 8 to 22.

5. The composition according to claim 1, wherein the non-ionic surfactant is selected from the group consisting of glyceryl oleate, glyceryl monostearate, glyceryl monoisostearate, glyceryl monopalmitate, glyceryl monobehenate, polyglyceryl-3 pentaricinoleate, polyglyceryl-6 pentaricinoleate, polyglyceryl-10 pentaricinoleate, polyglyceryl-3 polyricinoleate, polyglyceryl-6 polyricinoleate, polyglyceryl-3 ricinoleate, polyglyceryl-2 isostearate, polyglyceryl-4 isostearate, polyglyceryl-4 diisostearate/polyhydroxystearate/sebacate, polyglyceryl-5 isostearate, polyglyceryl-6 isostearate, polyglyceryl-10 isostearate, polyglyceryl-2 diisostearate, polyglyceryl-3 diisostearate, polyglyceryl-4 diisostearate, polyglyceryl-10 diisostearate, polyglyceryl-2 tetraisostearate, polyglyceryl-2 triisostearate, polyglyceryl-3 triisostearate, and mixture thereof.

6. The composition according to claim 1, wherein the non-ionic surfactant is present in an amount ranging from 0.1 wt. % to 30 wt. %, relative to the total weight of the composition.

7. The composition according to claim 1, wherein the polyethylene wax is present in an amount ranging from 3 wt. % to 80 wt. %, relative to the total weight of the composition.

8. The composition according to claim 1, further comprising a pasty compound selected from the group consisting of esters having the following INCI nomenclature: polyglyceryl-2 isostearate dimer dilinoleate copolymer, bis-behenyl/isostearyl/phytosteryl dimer dilinoleyl dimer dilinoleate, and mixtures thereof.

9. The composition according to claim 1, further comprising a thickener.

10. The composition according to claim 1, wherein the oil is selected from the group consisting of canola oil, squalane, pentaerythrityl tetraisostearate, caprylic/capric triglyceride, and mixtures thereof.

11. The composition according to claim 1, wherein the oil is present in an amount ranging from 10 wt. % to 90 wt. % relative to the total weight of the composition.

12. The composition according to claim 1, further comprising at least one colorant selected from the group consisting of water-soluble or water-insoluble, liposoluble or non-liposoluble, organic or inorganic colorants, and materials with an optical effect, and mixtures thereof.

13. The composition according to claim 12, wherein the colorant is present in an amount ranging from 0.01 wt. % to 35 wt. % relative to the total weight of the composition.

14. The composition according to claim 1 comprising, relative to the total weight of the composition:
a) from 4 wt. % to 10 wt. % of glycerin;
b) from 1 wt. % to 8 wt. % of at least one non-ionic surfactant selected from the group consisting of glyceryl monostearate, polyglyceryl-6 polyricinoleate, polyglyceryl-4 diisostearate/polyhydroxystearate/sebacate, polyglyceryl-4 isostearate, and mixture thereof;
c) from 5 wt. % to 15 wt. % of polyethylene wax;
d) from 40 wt. % to 80 wt. % at least one oil selected from the group consisting of canola oil, squalane, pentaerythrityl tetraisostearate, caprylic/capric triglyceride, mixtures thereof; and
e) from 5 wt. % to 15 wt. % of a colorant selected from the group consisting of YELLOW 6 LAKE, Blue 1 lake, RED 28 LAKE, RED 21, RED 7, titanium dioxide, iron oxides, synthetic fluorphlogopite, and mixtures thereof.

15. A cosmetic process for caring for and/or making up keratin materials comprising applying the composition as defined in claim 1, to the keratin materials.

16. The composition according to claim 4, wherein the non-ionic surfactant is selected from the group consisting of esters of saturated or unsaturated fatty acids having 16 to 22 carbon atoms and polyglycerin with a polyglyceryl moiety derived from 2 to 10 glycerol units.

17. The composition according to claim 9, wherein the thickener is a mineral thickener selected from the group consisting of silica silylate, fumed silica, zeolite, natural clay, synthetic clay, kaolin, hectorite, disteardimonium hectorite, stearalkonium hectorite, quaternium-18 bentonite, quaternium-18 hectorite, benzalkonium bentonite, and a mixture thereof
